(19) United States
(12) Patent Application Publication
(10) Pub. No.: US 2003/0017516 A1
(43) Pub. Date: Jan. 23, 2003

(54) Targeting tumor cell antigens: antibodies useful for the diagnosis, prognosis, and treatment of cancer

(75) Inventor: Ira M. Herman, Newton, MA (US)

Correspondence Address: LAHIVE & COCKFIELD, 28 STATE STREET, BOSTON, MA, 02109, US

(73) Assignee: Trustees of Tufts College, Boston, MA

(21) Appl. No.: 10/183,187

(22) Filed: Jun. 25, 2002

Related U.S. Application Data
(60) Provisional application No. 60/301,073, filed on Jun. 26, 2001.

(51) Int. Cl. G01N 33/574

(52) U.S. Cl. 435/7.23

(57) ABSTRACT

The invention features a novel tumor-associated monoclonal antibody which recognizes the mitosis-associated antigen, 4F2. The invention provides methods and antibodies for identifying precancerous cells, mitotic cells, and displastic regions of tumors. The invention further provides methods, kits, and antibodies for inhibiting the proliferation of precancerous and/or endothelial cells, inhibiting tumor derived angiogenesis, inhibiting disorders associated with unwanted endothelial cell proliferation, and detecting the presence of tumor cells in a subject.

### RELATED APPLICATIONS

The present application claims priority from U.S. provisional patent application serial No. 60/301,073, filed on Jun. 26, 2001, which is expressly incorporated by reference.

### GOVERNMENT SUPPORT

This invention was made with government support under NIH EY09033, NIH GM55110, and 5P30 DK34928 awarded by the National Institutes of Health to IMH. The government has certain rights in the invention.

### BACKGROUND OF THE INVENTION

Cell growth is mediated by the concerted action of numerous positive and negative factors. The decision to progress through the cell cycle is driven by cyclin/cdk (cyclin dependent kinase) complexes in the nucleus that phosphorylate key regulators such as the Rb (retinoblastoma) gene product to enable transcription of growth-promoting genes (Cordon-Cardo (1995) Am. J. Pathol. 147:545-560; Cobrinik et al. (1992) TIBS 17:312-315; and Johnson et al. (1993) Nature 365:349-352). Cdk inhibitors such as p21/WAF1/Cipl negatively regulate cyclin/cdk activity (Xiong et al. (1993) Nature 365:349). In the cytoplasm signal transduction via numerous pathways, including those activated by MAP and PI-3 kinases, relay both stimulatory and inhibitory cues from the plasma membrane to nuclear effectors (Davis (1993) J. Biol. Chem. 268:14553-14556; Toker et al. (1997) Nature 387:673-676). Growth control signals originate at the plasma membrane with cytokine receptors, adhesion molecules, and integrins, which receive extracellular stimuli and transmit regulatory signals to cytoplasmic signaling components (Goustin et al. (1986) Cancer Res. 46:1015-1029; Rosales et al. (1995) Biochim. Biophys. Acta. 1242:77-98; and Clark et al. (1995) Science 268:233-239). Exquisite control over all these regulatory molecules ensures maintenance of normal cell growth.

When control over cell growth is no longer maintained (e.g., in cancer), persistent positively acting signals are produced, and unbridled proliferation ensues. This unchecked growth is often the result of overexpression of key growth-promoting molecules, including the products of oncogenes such as Ras and Mdm2, and the mutation of growth-inhibiting factors such as the tumor suppressors p53 and APC (Bos (1989) Cancer Res. 49:4682-4689; Momand et al. (1992) Cell 69:1237-1245; Baker et al. (1990) Science 249:912-914; and Kinzler et al. (1996) Cell 87:159-170). In addition, alteration in cell-surface components often correlates with a tumorigenic cell phenotype.

For example, overexpression of the p185 neu/c-erbB-2 receptor has been reported in various human cancers, and induction of a deletion mutant of the EGF receptor in mouse fibroblasts results in an EGF-independent transformed phenotype (Dougall et al. (1994) Oncogene 9:2109-2123; Khazaie et al. (1988) EMBO J. 7:3061-3071). Because many cell surface alterations are results of tumor progression, they have been characterized as tumor specific (Carraway et al. (1992) J. Cell Science 103:299-307).

Tumor-specific cell surface antigens have been described in many different tissues (reviewed in Virgi et al. (1988) CA Cancer J. Clin. 38:104-127; Pastan et al. (1996) Breast Cancer Research And Treatment 38:3-9; Carraway et al. (1992) J. Cell Science 103:299-307; and Wick et al. (1997) Cancer Lett. 118:161-172). For example, carcinomas of the lung, breast, colon, and ovary show abundant L6 surface antigen, while normal cells demonstrate only limited expression (Hellstrom et al. (1986) Cancer Res. 46:3917-3923; Marken et al. (1992) Proc. Nat. Acad. Sci. USA 3503-3507). Mucinous carcinomas of the colon, stomach, and ovary, highly express the carbohydrate antigens recognized by tumor-specific monoclonal antibodies B1 and B3 (Pastan et al. (1991) Cancer Res. 51:3781-3787). Human breast tumor is the source of the 85 kDa glycoprotein BTAA to which circulating antibodies were discovered in breast cancer patients but not in normal women or patients with other carcinomas (Pal et al. (1995) Int. J. Cancer 50:759-765). In prostate tissue, several tumor-specific antigens have been identified using antibodies isolated from mice immunized with prostate tumor cells or cell membranes (Beckett et al. (1991) Cancer Res. 51:1326-1333; Tjota et al. (1991) J. Virol. 146:205-212; and Pastan et al. (1993) J. Natl. Cancer Inst. 85:1149-1154). Both ductal epithelia and secretions of prostate adenocarcinoma are highly enriched in the mucin-like antigen recognized by the monoclonal antibody PD41, while fetal or benign prostate specimens are devoid of this antigen (Beckett et al., supra). Androgen-independent rat prostate tumor cell lines and human prostate carcinoma tissue, but not normal rat or human tissues or benign prostatic hyperplasia tissue, express cell surface and cytoplasmic antigens of 50 and 120 kDa recognized by monoclonal antibody MCA-R1 (Tjota et al., supra). Clearly, in a variety of cancers there appears to be expression of cell-surface antigens that are correlated with a tumorigenic phenotype.

Targeting of tumor-specific cell surface proteins with antibodies or with immunotoxins in order to eradicate tumors has demonstrated some success (Pastan et al. (1986) Cell 47:641-648). For example, an immunotoxin to mesothelin, a differentiation antigen on the surface of mesotheliomas as well as ovarian and other human cancers, demonstrates high cytotoxicity to mesothelin-expressing cells, and causes regression of mesothelin-expressing subcutaneous tumors in immunodeficient mice (Chang et al. (1992) Cancer Res. 52:181-186; Chowdhury et al. (1998) Proc. Natl. Acad. Sci. USA 95:669-674). An immunotoxin against the IL-2 receptor, which is expressed on the surface of many leukemias and lymphomas but not on normal resting T cells, causes complete regression of IL-2 receptor-bearing subcutaneous tumor xenographs (Waldmann (1986) Science 232:727-732; Reiter et al. (1994) Int. J. Cancer 58:142-149). Furthermore, an immunotoxin comprised of IL-4 fused to a fragment of Pseudomonas exotoxin substantially reduces or completely eliminates established subcutaneous AIDS KS tumors in immunodeficient mice in a dose-dependent manner (Husain et al. (1999) Nature Med. 5:817-821). Limited success has been attained in phase I clinical trials of immunotoxins. The RFB4 immunotoxin, which targets CD22, mediated partial remission of tumors in 40% of treated B-cell lymphoma patients, and the LMB-1 immunotoxin which utilizes the B3 antibody described above significantly reduced epithelial tumors in 5 of 38 patients who failed conventional therapy (Vitetta et al. (1991) Cancer Res. 51:4052-4058; Pai et al. (1996) Nature Med. 2:350-353). The importance and clinical efficacy of targeting these tumor-associated antigens has thus been demonstrated. Enrichment of tumors vs. normal tissues with these antigens defines cancerous cell targets, and expression of these antigens on the cell surface makes them highly accessible to tumor-specific antibodies and immunotoxins.

Although considerable progress has been made in identifying proteins enriched in tumor populations which can be used as therapeutic targets for cancer treatment, only limited success has been achieved in eradicating tumors in the clinic (Vitetta et al. and Pai et al., supra). At best, only partial remission or significant reduction of human tumors has been achieved with therapeutic agents such as immunotoxins in only a subset of treated patients. The limited efficacy of these tumor immunotoxins in treating human cancer demonstrates the need to find an alternative, more effective strategy for targeting and destroying cancerous cells.

Another approach to finding a tumor-specific antigen is to relate its presence on a cancer cell in a functional sense to a fundamental aspect of a tumor. A protein that is highly expressed during mitosis, for example, is functionally associated with a tumor cell because of its capacity for uncontrolled cell division. The importance of mitosis-specific protein regulation is underscored by the HER2-neu growth factor receptor. Similar to the EGF receptor, HER2-neu shows markedly decreased tyrosine kinase activity in mitosis coincident with hyper-phosphorylation on serine and threonine residues (Kiyakowa et al. (1997) J. Biol. Chem. 272:18656-18665; Kiyokowa et al. (1995) Proc. Natl. Acad. Sci. USA 92:1092-1096). Moreover, a point mutation in HER2-neu which renders it unresponsive to this mitosis-specific regulation generates a protein with potent transforming ability. Mitosis-specific protein modification thus has important consequences for normal cell growth.

### SUMMARY OF THE INVENTION

A novel monoclonal antibody, hereinafter designated anti-βE11 or βE11, recognizes an antigen that is highly abundant on the surface of mitotic endothelial cells as well as various tumor cells. The βE11 antibody inhibits the growth of tumor cells and, to a limited extent, endothelial cells, in a dose-dependent manner in vitro. Furthermore, the βE11 antigen is identical to the 4F2 antigen, a glycoprotein originally characterized to be expressed on the surface of activated, but not resting, lymphocytes and shown to be associated with cell proliferation (Eisenbarth et al. (1980) J. Immunol. 124:1237-1244; Haynes et al. (1981) J. Immunol. 126:1409-1414; and Luscher et al. (1985) J. Immunol. 135:3951-3957).

The present invention provides a novel approach for targeting an antigen associated with mitosis and proliferation in order to inhibit tumor cell progression. In contrast to approaches used in the art to target proteins enriched in tumor populations, the present invention teaches the identification and inhibition of cancerous and precancerous cells by the modification (i.e., inhibition) of proteins which are mitosis-specific. This novel approach has the added benefit being applicable to the attenuation of angiogenesis, since the anti-4F2 antibodies of the invention are capable of inhibiting growth of primary endothelial cells. Furthermore, this approach has the added benefit of targeting an antigen which is expressed at the cell surface and is, therefore, readily accessible to the antibodies of the present invention.

In one embodiment, the invention provides a method of identifying a precancerous cell. The method includes contacting a precancerous cell with an antibody that binds to a 4F2 antigen present on a precancerous cell, such that binding of the antibody to a cell identifies the cell as a precancerous cell. In a preferred embodiment, the precancerous cell is obtained from a biological sample. In another preferred embodiment, the biological sample is tissue, cells, blood, serum, urine, feces, and/or saliva. In another preferred embodiment, the precancerous cell is obtained from a biopsy sample. In another preferred embodiment, the antibody is a monoclonal antibody. In a particularly preferred embodiment, the monoclonal antibody is anti-βE11.

In another embodiment, the invention provides a method of distinguishing mitotic cells from cells in interphase. The method includes contacting a population of cells comprising at least one mitotic cell and at least one cell in interphase with an antibody that binds to a 4F2 antigen, such that binding of the antibody to a cell identifies the cell as a mitotic cell. The method also includes detecting the binding of the antibody to a cell of the cell population. The method further includes identifying the cell that binds the antibody as a mitotic cell to thereby distinguish mitotic cells from cells in interphase. In a preferred embodiment, the cells are obtained from a biological sample. In another preferred embodiment, the biological sample is tissue, cells, blood, serum, urine, feces, and/or saliva. In another preferred embodiment, the cells are obtained from a biopsy sample. In another preferred embodiment, the antibody is a monoclonal antibody. In a particularly preferred embodiment, the monoclonal antibody is anti-βE11.

In another embodiment, the invention provides a method of detecting displastic regions of a tumor by contacting a tumor with an antibody that binds to a 4F2 antigen such that displastic regions of a tumor are detected. In a preferred embodiment, the tumor is obtained from a biological sample. In another preferred embodiment, the tumor is obtained from a biopsy sample. In another preferred embodiment, the antibody is a monoclonal antibody. In a particularly preferred embodiment, the monoclonal antibody is anti-βE11.

In another embodiment, the invention provides a method of inhibiting proliferation of a precancerous cell by contacting a precancerous cell with an antibody that binds to a 4F2 antigen such that proliferation of a precancerous cell is inhibited. In a preferred embodiment, the antibody is a monoclonal antibody. In another preferred embodiment, the monoclonal antibody is anti-βE11. In another preferred embodiment, the antibody is administered to a subject in a pharmaceutically acceptable formulation. In a particularly preferred embodiment, the subject is human.

In another embodiment, the invention provides a method of inhibiting proliferation of a precancerous cell by contacting a precancerous cell with an antibody that binds to a 4F2 antigen and a low molecular weight inhibitor such that proliferation of a precancerous cell is inhibited. In a preferred embodiment, the antibody is a monoclonal antibody. In another preferred embodiment, the monoclonal antibody is anti-βE11. In another preferred embodiment, the antibody is administered to a subject in a pharmaceutically acceptable formulation. In a particularly preferred embodiment, the subject is human.

In another embodiment, the invention provides a method of inhibiting a disorder associated with endothelial cell growth by contacting an endothelial cell with an antibody that binds to a 4F2 antigen such that the disorder is inhibited. In a preferred embodiment, the antibody is a monoclonal antibody. In another preferred embodiment, the monoclonal antibody is anti-βE11. In another preferred embodiment, the antibody is administered to a subject in a pharmaceutically acceptable formulation. In a particularly preferred embodiment, the subject is human. In another particularly preferred embodiment, the disorder is selected from the group consisting of diabetic retinopathy, age related macular degeneration, chronic inflammatory disorders, and ovarian disorders.

In another embodiment, the invention provides a method of inhibiting tumor derived angiogenesis by contacting an endothelial cell with an antibody that binds to a 4F2 antigen such that proliferation of an endothelial cell is inhibited. In a preferred embodiment, the antibody is a monoclonal antibody. In another preferred embodiment, the monoclonal antibody is anti-βE11. In another preferred embodiment, the antibody is administered to a subject in a pharmaceutically acceptable formulation. In a particularly preferred embodiment, the subject is human.

In another embodiment, the invention provides a method of detecting the presence of tumor cells in a patient by testing for the presence of a 4F2 antigen in a blood sample of the patient. In a preferred embodiment, the tumor cells are selected from the group consisting of colon tumor cells, glioma cells, melanoma cells, fibroblast tumor cells, breast tumor cells, and liver tumor cells. In a particularly preferred embodiment, the tumor cells are prostate tumor cells.

In another preferred embodiment, the invention provides a kit for the diagnosis and/or prognosis of prostate cancer. The kit includes an antibody that recognizes the 4F2 antigen and instructions for use.

In still another embodiment, the invention provides a method for facilitating the prognosis of a disorder associated with endothelial cell growth in a subject. In a preferred embodiment, a 4F2 antigen is detected in a biological sample from a subject with an antibody that binds to a 4F2 antigen, thereby facilitating the prognosis of a disorder associated with endothelial cell growth in a subject. In another preferred embodiment, the biological sample is selected from the group consisting of tissue, cells, blood, serum, urine, feces, and saliva. In another preferred embodiment, the sample is obtained from a biopsy sample. In a preferred embodiment, the antibody is a monoclonal antibody. In another preferred embodiment, the monoclonal antibody is anti-βE11. In still another preferred embodiment, the disorder is selected from the group consisting of diabetic retinopathy, age related macular degeneration, chronic inflammatory disorders, and ovarian disorders.

In yet another preferred embodiment, the invention provides an antibody (e.g., a 4F2 antibody, e.g., a β11 antibody).

### BRIEF DESCRIPTION OF THE DRAWINGS

FIGS. 1A-1P depict surface immunostaining of mitotic vascular endothelial cells and tumor cells with anti-βE11. A-H depict anti-βE11 stains of the surface of endothelial cells during mitosis. A, C, E, and G depict anti-βE11 immunofluorescence of formaldehyde-fixed, non-permeabilized bovine retinal endothelial cells. B, D, and F depict paired Hoescht-stained images. Cells in metaphase, anaphase, and cytokinesis are stained by anti-βE11, while surrounding interphase cells are not. I-N depict anti-β-E11 staining of both mitotic and interphase cells in the human prostate carcinoma cell line LNCaP. J and L depict paired Hoescht-stained images. M and N depict permeabilized myoblast cell line C2 cells stained with anti-βE11 and the paired Hoescht-stained image, respectively. M and N indicate that anti-βE11 stains C2 myoblasts during mitosis but not during interphase. O and P depict avidin-biotin peroxidase immunohistochemistry performed on frozen prostatic tumor tissue sections with anti-βE11 (O) or a murine IgG control (P). O and P indicate that anti-βE11 recognizes displastic regions of prostate tumor tissue.

FIGS. 2A-2B depict Western blots of the βE11 antigen in primary endothelial cells and tumor-derived cell lines. A depicts a Western blot of approximately 500,000 cell equivalents from detergent lysates of bovine retinal endothelial cells at approximately 50% confluency (lane 2) or growth arrested in a monolayer (lane 1) probed with ant-βE11 followed by goat anti-mouse IgG-HRP (immunoglobulin G-horseradish peroxidase). B depicts approximately 15 μg of tumor cell lysate blotted with anti-βE11 followed by goat anti-mouse IgG-HRP (top panel). The corresponding gel stained with Coomassie Blue is depicted in the middle panel. Lane 1, LNCaP human prostate; lane 2, Cx.1 human colon carcinoma; lane 3, murine endothelioma; lane 4, Lewis lung; lane 5, murine prostate; lane 6, rat glioma; lane 7, murine melanoma; and lane 8, HEPG2 human liver. Relative protein levels loaded on the gel and relative expression levels of the βE11 antigen were determined by scanning densitometry of Western blots (bottom panel). βE11 antigen expression was normalized by dividing the expression level by protein level and arbitrarily assigning the HEPG2 cell lysate a value of 1.

FIGS. 3A-3D depict the inhibition of tumor cell growth in vitro. Primary and tumor cells were grown in the presence or absence of anti-βE11 or a murine IgG control for the number of days indicated. A depicts human colon carcinoma cells. B depicts LNCaP human prostate tumor cells. C depicts LNCaP cells which were grown as in B for three days, after which time antibody was washed out of the medium (indicated by the arrow). D summarizes the maximal inhibition observed for each cell line used in the growth assays. For all cell lines, maximal inhibition occurred at days five to seven at an anti-βE11 concentration of 0.5-1.0 μg/mL. From top to bottom, cell lines are as follows: primary bovine retinal endothelial cells; murine embryonic endothelioma cells; murine brain endothelioma cells; primary bovine retinal pericytes; Lewis lung carcinoma cells; B16 BL6 murine melanoma cells; LNCaP human prostate cells; rat prostate cells; human urinary bladder carcinoma cells; Cx.1 human colon carcinoma cells; murine 3T3 fibroblasts; human astrocytoma cells; C6 rat glioma cells; HEPG2 human liver carcinoma cells; and MSCF7 human breast carcinoma cells.

FIGS. 4A-4B depict the expression of the βE11 antigen in serum-free melanoma cells. A depicts a Western blot of detergent lysates, containing approximately 10 μg total protein, obtained from LNCaP cells grown in serum (lane 1) and B16 BL6 melanoma cells grown in the presence (lane 2) or absence (lane 3) of serum. The Western was probed with anti-βE11 followed by goat anti-mouse IgG-HRP. B depicts immunoprecipitation of lysates obtained from LNCaP cells grown in serum (lane 1) or from melanoma cells grown in the absence of serum (lane 2). The immunoprecipitation was performed using serum-free affinity isolated anti-βE11 and goat anti-mouse IgG (F_{c})-sepharose. The immunoprecipitated complexes were separated on an 8.5% SDS-PAGE (sodium dodecyl sulfate-polyacrylamide gel electrophoresis) gel and transferred to a nitrocellulose membrane. The blots were probed with anti-βE11 followed by goat anti-mouse IgG-HRP. The double asterisk indicates the IgG heavy chain.

FIGS. 5A-5B depict two-dimensional gel electrophoresis and mass spectrometry of the βE11 antigen. Approximately 125 μg of total protein from melanoma cells grown in the absence of serum was separated by two-dimensional PAGE. Isoelectric focusing in the presence of SDS from pH 3 to pH 7 was performed in the first dimension, while the second dimension was performed in a 5% SDS-PAGE gel under reducing conditions. A depicts a gel stained with Coomassie Blue. B depicts a corresponding Western blot probed with anti-βE11 followed by goat anti-mouse IgG-HRP. Spots 1 and 2 were excised from the gel stained with Coomassie Blue, and were processed for mass spectrometry as described below.

FIGS. 6A-6B depict a direct comparison of antigens recognized by anti-βE11 and anti-4F2. A depicts B16 BL6 melanoma cells which were grown in serum-free medium and lysed in sample buffer containing 20% SDS and 10% β-mercaptoethanol. Western blotting was performed on approximately 15 μg of lysate using either anti-βE11 (lane 1) or anti-4F2 (lane 2) as a probe. B depicts a V8 protease limited digest comparison of the βE11 and 4F2 antigens. Complexes immunoprecipitated by anti-βE11 (A) or anti-4F2 (B) from radiolabeled melanoma cells which were grown in serum-free medium were incubated for five minutes with the indicated amount of Staphylococcus V8 protease. The peptides generated after digestion were separated on a 10% SDS-PAGE gel under reducing conditions and visualized by autoradiography.

FIGS. 7A-7D depict a pulse-chase analysis of the βE11 and 4F2 antigens. B16 BL6 melanoma cells that were cultured in serum-free medium were pulse-labeled for 2 hours and chased for the time periods indicated. Panels A and B were immunoprecipitated with anti-βE11, and panels C and D were immunoprecipitated with anti-4F2. Immunoprecipitates were analyzed using an 8.5% SDS-PAGE gel under reducing conditions, and the gels were processed by autoradiography. In panels B and D the cells were pre-incubated with tunicamycin for eighteen hours.

FIGS. 8A-8B depict a pulse-chase analysis of βE11 and 4F2 antigens. B16 BL6 melanoma cells cultured in serum-free medium (Biowhittaker, Walkersville, Md.) were pretreated with tunicamycin for eighteen hours. The cells were then pulse-labeled for two hours and chased for the additional time periods indicated. Anti-βE11 (A) or anti-4F2 (B) immunoprecipitates were run on reducing 8.5% SDS-PAGE, and the gels were processed for autoradiography.

### DETAILED DESCRIPTION OF THE INVENTION

The present invention features a novel tumor-associated monoclonal antibody hereinafter designated βE11, which is characterized as recognizing a mitosis-specific antigen (e.g., 4F2). The invention provides methods and antibodies for identifying precancerous cells, mitotic cells, and displastic regions of tumors. The invention further provides methods, kits, and antibodies for inhibiting the proliferation of precancerous cells, inhibiting tumor derived angiogenesis, inhibiting disorders associated with unwanted endothelial cell growth, and detecting the presence of tumor cells in a subject. Whereas previous work has focused on targeting tumor-specific cell surface proteins, the present invention offers a novel approach to the inhibition of tumor progression. The methods of the invention permit the inhibition of proteins specific to mitosis. Since tumor cells undergo uncontrolled cell division, inhibiting proteins functionally associated with cell division can inhibit tumor cell growth.

The anti-4F2 antibodies of the present invention were identified based on their ability to bind an antigen that is highly abundant on the surface of mitotic endothelial cells as well as many tumor cells. Accordingly, the anti-4F2 molecules may act as novel diagnostic and/or prognostic targets and therapeutic agents for controlling cancerous and/or precancerous cells. The anti-4F2 antibodies (e.g., anti-βE11 antibodies) of the present invention may also regulate angiogenesis by modulating the proliferation of endothelial cells that extend the vascular network to deliver nutrients to tumor cells. The anti-4F2 antibodies (e.g., anti-βE11 antibodies) of the present invention are useful for treating disorders associated with unwanted endothelial cell growth including, but not limited to: 1) tumor cell proliferation; 2) diabetic retinopathy; 3) age related macular degeneration; 4) chronic inflammatory disorders (e.g., psoriasis and rheumatoid arthritis); and 5) ovarian disorders (e.g., anovulation and infertility, pregnancy loss, ovarian hyperstimulation syndrome, and ovarian neoplasms). Thus, the anti-4F2 antibodies of the present invention are useful for the prevention of tumor cell proliferation and/or angiogenesis-related disorders.

Isolated antibodies of the present invention, preferably anti-4F2 (e.g., anti-βE11) antibodies, share a common functional activity are defined herein as sufficiently identical. As used interchangeably herein, a “βE11 activity”, “anti-βE11 activity”, “anti-4F2 activity”, “biological activity of anti-βE11”, “biological activity of anti-4F2”, “functional activity of anti-βE11” or “functional activity of anti-4F2” refers to an activity exerted by an anti-βE11 and/or anti-4F2 antibody on a 4F2 responsive cell or tissue, or on a 4F2 antigen, as determined in vivo, or in vitro, according to standard techniques. In one embodiment, an anti-4F2 activity is a direct activity, such as binding a 4F2 antigen. As used herein, a “4F2 antigen” and/or “4F2 protein” is a molecule with which an anti-4F2 antibody binds or interacts in nature, such that anti-4F2-mediated function is achieved. Alternatively, an anti-4F2 target molecule can be a non-4F2 protein or polypeptide of the present invention. In an exemplary embodiment, an anti-4F2 target molecule is a 4F2 substrate, e.g., a mitosis-specific protein. Alternatively, an anti-4F2 activity is an indirect activity mediated by interaction of the anti-4F2 antibody with a 4F2 substrate, e.g., a mitosis-specific protein. Preferably, an anti-4F2 activity is the ability to act as a 4F2-binding factor and to modulate functions such as tumor proliferation and angiogenesis. Accordingly, another embodiment of the invention features isolated anti-4F2 antibodies (e.g., anti-βE11 antibodies) having an anti-4F2 activity. Preferred antibodies are anti-4F2 antibodies having an anti-4F2 activity.

Various aspects of the invention are further described below.

I. Definitions

As used herein, the term “displastic” is intended to include an abnormality of development in pathology, alteration in size, shape, and organization of adult cells.

As used herein, the term “angiogenesis” is intended to include the process of vascularization of a tissue involving the development of new capillary blood vessels. The term “proliferation” is intended to include the reproduction or multiplication of cells.

The term “mitotic”, as used herein, is intended to include the method of indirect division of a cell, consisting of a complex of various processes, by means of which the two daughter nuclei normally receive identical complements of the number of chromosomes characteristic of the somatic cells. Mitosis, the process by which the body grows and replaces cells, is divided into four phases, prophase, metaphase, anaphase, and telophase. The terms “interphase” and “non-mitotic” are intended to include the stage of a cell when it is not in mitosis, hence comprising most of the cell cycle.

The terms “4F2 antigen” and “βE11 antigen”, as used herein, are intended to include a type II glycoprotein of approximately 190 kDa which was originally characterized to be expressed on the surface of activated, but not resting, lymphocytes and shown to be associated with cell proliferation. The 4F2 antigen is described in more detail in the references Parmeck et al. (1989) Nucl. Acid Res. 17:1915-1931 and Azzarone et al. (1985) Exp. Cell Res. 159:451-462, incorporated herein by reference.

As used herein, the term “biological sample” is intended to include tissues, cells and biological fluids isolated from a subject, as well as tissues, cells and fluids present within a subject.

As used herein, the terms “proliferation of a precancerous cell” and “tumor derived angiogenesis” are intended to include a disease or disorder that affects a cell growth or proliferation process. As used herein, a “cellular growth or proliferation process” is a process by which a cell increases in number, size or content, by which a cell develops a specialized set of characteristics which differ from that of other cells, or by which a cell moves closer to or further from a particular location or stimulus. A cellular growth or proliferation process includes the metabolic processes of the cell and cellular transcriptional activation mechanisms. A cellular growth or proliferation disorder may be characterized by aberrantly regulated cell growth, proliferation, differentiation, or migration. Cellular growth or proliferation disorders include tumorigenic disease or disorders. As used herein, a “tumorigenic disease or disorder” includes a disease or-disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of cellular growth or proliferation disorders include, but are not limited to, cancer, e.g., carcinoma, sarcoma, or leukemia, examples of which include, but are not limited to, colon, ovarian, lung, breast, endometrial, uterine, hepatic, gastrointestinal, prostate, and brain cancer; tumorigenesis and metastasis; skeletal dysplasia; and hematopoietic and/or myeloproliferative disorders.

The term “unwanted endothelial cell growth” as used herein, is intended to include diseases associated with the proliferation of endothelial cells. Such diseases include, but are not limited to, tumor cell proliferation, diabetic retinopathy, age related macular degeneration, chronic inflammatory disorders (e.g., psoriasis and rheumatoid arthritis), and ovarian disorders (e.g., anovulation and infertility, pregnancy loss, ovarian hyperstimulation syndrome, and ovarian neoplasms).

As used herein, the term “precancerous” is intended to include a very early stage of cancer development during which time changes occur in a cell and/or tissue as a predeterminant for possible future malignancy. The term “malignancy”, as used herein, is intended to include diseases that are characterized by uncontrolled, abnormal growth of cells.

The term “subject”, as used herein, refers to a human subject who has presented at a clinical setting with a particular symptom or symptoms suggesting one or more diagnoses. A subject may be in need of further categorization by clinical procedures well-known to medical practitioners of the art (or may have no further disease indications and appear to be in any or all respects normal). A subject's diagnosis may alter during the course of disease progression, such as development of further disease symptoms, or remission of the disease, either spontaneously or during the course of a therapeutic regimen or treatment. In the present invention, a subject described in the Examples is listed with other patients according to the most recent diagnosis of the medical condition, and any previous diagnoses, if different, are described in the text. Thus, the term “diagnosis” does not preclude different earlier or later diagnoses for any particular patient or subject. The term “prognosis” refers to assessment for a subject or patient of a probability of developing a condition associated with or otherwise indicated by presence of one or more enzymes in a biological sample.

II. Anti-4F2 Pharmaceutical Compositions

In one aspect, the invention is drawn to methods of contacting a cell with an anti-4F2 antibody (e.g., anti-βE11) in vitro or in vivo in order to inhibit the proliferation of a precancerous cell, inhibit unwanted proliferation of an endothelial cell, and/or to inhibit tumor derived angiogenesis. The anti-4F2 antibodies (e.g., anti-βE11) (also referred to herein as “active compounds”) of the invention can be incorporated into pharmaceutical compositions suitable for administration. Such compositions typically comprise the anti-4F2 antibody (and optionally, an art-known low molecular weight inhibitor) and a pharmaceutically acceptable carrier. As used herein, the term “low molecular weight inhibitor” includes toxins which, when taken up by cells, result in cell death. Low molecular weight inhibitors (e.g., neoplastic drugs) are art recognized and include, but are not limited to cisplatin, colchicine, chlorambucil, carboplatin, bleomycin, CT-2519 (1-(5-isothiocyanatohexyl)-3,7-dimethylxanthine), hydroxyurea, PSI (carbobenzoxy-Lisoleucyl-gamma-t-butyl-L-glutamyl-L-alanyl-L-leucinal), small molecule tyrosine kinase inhibitors (e.g., inhibitors from the quinazoline and pyrazolo-pyrrolo-pyridopyrimidine inhibitor structural classes), and cyclin-dependent kinase inhibitors (e.g., inhibitors directed against the ATP binding sites of cdk1, cdk2, and cdk4). As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the calpain inhibitor) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic and/or prognostic assays as described herein.

The pharmaceutical compositions can be included in a container, pack, or dispenser together with instructions for administration and treatment.

III. Anti-4F2 Antibodies

Given their ability to bind to tumor cells and inhibit tumor growth, as well as the cell surface location of the 4F2 antigen, the anti-4F2 antibodies, or portions thereof, of the invention can be used to detect cancerous and/or precancerous cells (e.g., in a biological sample, such as serum or plasma), using a conventional immunoassay, such as an enzyme linked immunosorbent assays (ELISA), an radioimmunoassay (RIA) or tissue immunohistochemistry. The invention provides a method for detecting cancerous and/or precancerous cells in a biological sample comprising contacting a biological sample with an antibody, or antibody portion, of the invention and detecting either the antibody (or antibody portion) bound to 4F2 or unbound antibody (or antibody portion), to thereby detect cancerous and/or precancerous cells in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Alternative to labeling the antibody, cancerous (i.e., tumor derived) and/or precancerous cells can be assayed in biological fluids by a competition immunoassay utilizing non-cancerous cell standards labeled with a detectable substance and an unlabeled anti-4F2 antibody. In this assay, the biological sample, the labeled non-cancerous cell standards and the anti-4F2 antibody are combined and the amount of labeled non-cancerous cell standard bound to the unlabeled antibody is determined. The amount of cancerous and/or precancerous cells in the biological sample is inversely proportional to the amount of labeled non-cancerous cell standard bound to the anti-4F2 antibody.

In another embodiment, the invention provides a method for inhibiting unwanted endothelial cell growth (e.g., inhibiting proliferation of a precancerous cell and/or inhibiting tumor derived angiogenesis), comprising administering to the subject an antibody or antibody portion of the invention such that proliferation and/or tumor derived angiogenesis in the subject is inhibited. Disorders associated with unwanted endothelial cell growth include, but are not limited to: 1) tumor cell proliferation; 2) diabetic retinopathy; 3) age related macular degeneration; 4) chronic inflammatory disorders (e.g., psoriasis and rheumatoid arthritis); and 5) ovarian disorders (e.g., anovulation and infertility, pregnancy loss, ovarian hyperstimulation syndrome, and ovarian neoplasms). Preferably, the subject is a human subject. An antibody of the invention can be administered to a human subject for therapeutic purposes. Moreover, an antibody of the invention can be administered to a non-human mammal expressing a cancerous and/or precancerous cell with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

An isolated 4F2 protein, or a portion or fragment thereof, can be used as an immunogen to generate antibodies that bind 4F2 using standard techniques for polyclonal and monoclonal antibody preparation. A full-length 4F2 protein can be used or, alternatively, the invention provides antigenic peptide fragments of 4F2 for use as immunogens. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 4F2 that are located on the surface of the protein, e.g., hydrophilic regions, as well as regions with high antigenicity.

A 4F2 immunogen typically is used to prepare antibodies by immunizing a suitable subject, (e.g., rabbit, goat, mouse or other mammal) with the immunogen. An appropriate immunogenic preparation can contain, for example, recombinantly expressed 4F2 protein or a chemically synthesized 4F2 polypeptide. The preparation can further include an adjuvant, such as Freund's complete or incomplete adjuvant, or similar immunostimulatory agent. Immunization of a suitable subject with an immunogenic 4F2 preparation induces a polyclonal anti-4F2 antibody response.

Accordingly, another aspect of the invention pertains to anti-4F2 antibodies. The term “antibody” as used herein refers to immunoglobulin molecules and immunologically active portions of immunoglobulin molecules, i.e., molecules that contain an antigen binding site which specifically binds (immunoreacts with) an antigen, such as 4F2. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 4F2. The term “monoclonal antibody” or “monoclonal antibody composition”, as used herein, refers to a population of antibody molecules that contain only one species of an antigen binding site capable of immunoreacting with a particular epitope of 4F2. A monoclonal antibody composition thus typically displays a single binding affinity for a particular 4F2 antigen with which it immunoreacts.

Polyclonal anti-4F2 antibodies can be prepared as described above by immunizing a suitable subject with a 4F2 immunogen. The anti-4F2 antibody titer in the immunized subject can be monitored over time by standard techniques, such as with an enzyme linked immunosorbent assay (ELISA) using immobilized 4F2. If desired, the antibody molecules directed against βE11 can be isolated from the mammal (e.g., from the blood) and further purified by well known techniques, such as protein A chromatography to obtain the IgG fraction. At an appropriate time after immunization, e.g., when the anti-4F2 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a 4F2 immunogen as described above, and the culture supernatants of the resulting hybridoma cells are screened to identify a hybridoma producing a monoclonal antibody that binds 4F2.

Any of the many well known protocols used for fusing lymphocytes and immortalized cell lines can be applied for the purpose of generating an anti-4F2 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 4F2, e.g., using a standard ELISA assay.

Alternative to preparing monoclonal antibody-secreting hybridomas, a monoclonal anti-4F2 antibody can be identified and isolated by screening a recombinant combinatorial immunoglobulin library (e.g., an antibody phage display library) with 4F2 to thereby isolate immunoglobulin library members that bind 4F2. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-4F2 antibodies, such as chimeric and humanized monoclonal antibodies, comprising both human and non-human portions, which can be made using standard recombinant DNA techniques, are within the scope of the invention, and are discussed in more detail below. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

IV. Expression of Antibodies

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express βE11 or a βE11-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{κ} Segments Reveals a Strong Bias in their Usage” Eur. J. Immunol. 24:827-836; the contents of each of which are expressly incorporated herein by reference). To obtain a DNA fragment encoding the heavy chain variable region of βE11, or a βE11-related antibody, a member of the V_{H}3 family of human germline VH genes is amplified by standard PCR. To obtain a DNA fragment encoding the light chain variable region of βE11, or a βE11-related antibody, a member of the V_{κ}I family of human germline VL genes is amplified by standard PCR. PCR primers suitable for use in amplifying the germline VH and germline VL sequences can be designed based on the nucleotide sequences disclosed in the references cited supra, using standard methods.

Once the germline VH and VL fragments are obtained, these sequences can be mutated to encode the βE11 or βE11-related amino acid sequences that can be determined by routine methods known to those of skill in the art. The amino acid sequences encoded by the germline VH and VL DNA sequences are first compared to the βE11 or βE11-related VH and VL amino acid sequences to identify amino acid residues in the βE11 or βE11-related sequence that differ from germline. Then, the appropriate nucleotides of the germline DNA sequences are mutated such that the mutated germline sequence encodes the βE11 or βE11-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Once DNA fragments encoding βE11 or βE11-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions of the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the βE11 or βE11-related light or heavy chain sequences, the expression vector may already carry antibody constant region sequences. For example, one approach to converting the βE11 or βE11-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” is intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr- CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

V. Diagnostic Assays

An exemplary method for detecting the presence or absence of a 4F2 antigen in a biological sample involves obtaining a biological sample from a test subject and contacting the biological sample with a compound or an agent (e.g., anti-4F2 antibodies) capable of detecting a 4F2 antigen such that the presence of a 4F2 antigen is detected in the biological sample. In another aspect, the present invention provides a method for detecting the presence of a 4F2 antigen activity in a biological sample by contacting the biological sample with an agent (e.g., anti-4F2 antibodies) capable of detecting an indicator of a 4F2 antigen activity such that the presence of a 4F2 antigen activity is detected in the biological sample.

A preferred agent for detecting a 4F2 antigen is an antibody capable of binding to a 4F2 antigen polypeptide, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect a 4F2 antigen in a biological sample in vitro as well as in vivo. For example, in vitro techniques for detection of a 4F2 antigen include enzyme linked immunosorbent assays (ELISAs), Western blots, immunoprecipitations and immunofluorescence. Furthermore, in vivo techniques for detection of a 4F2 antigen include introducing into a subject a labeled anti-4F2 antibody. For example, the antibody can be labeled with a radioactive marker whose presence and location in a subject can be detected by standard imaging techniques.

In one embodiment, the biological sample contains polypeptide molecules from the test subject. A preferred biological sample is a serum sample isolated by conventional means from a subject.

In another embodiment, the methods further involve obtaining a control biological sample from a control subject, contacting the control sample with a compound or agent capable of detecting a 4F2 antigen, such that the presence of a 4F2 antigen is detected in the biological sample, and comparing the presence of a 4F2 antigen in the control sample with the presence of a 4F2 antigen in the test sample.

The invention also encompasses kits for detecting the presence of a 4F2 antigen in a biological sample. For example, the kit can comprise a labeled compound or agent capable of detecting a 4F2 antigen in a biological sample; means for determining the amount of a 4F2 antigen in the sample; and means for comparing the amount of a 4F2 antigen in the sample with a standard. The compound or agent can be packaged in a suitable container. The kit can further comprise instructions for using the kit to detect a 4F2 antigen.

VI. Prognostic Assays

The diagnostic methods described herein can furthermore be utilized to identify subjects having or at risk of developing a disease or disorder associated with aberrant 4F2 antigen expression or activity. As used herein, the term “aberrant” includes a 4F2 antigen expression which deviates from the wild type a 4F2 antigen expression or activity. Aberrant expression or activity includes increased or decreased expression or activity, as well as expression or activity which does not follow the wild type developmental pattern of expression or activity or the subcellular pattern of expression or activity. For example, aberrant a 4F2 antigen expression or activity is intended to include the cases in which a 4F2 antigen is over-expressed.

The assays described herein, such as the preceding diagnostic assays or the following assays, can be utilized to identify a subject having or at risk of developing a disorder associated with a misregulation of a 4F2 antigen, such as disorders associated with unwanted endothelial cell growth (e.g., tumor cell proliferation; diabetic retinopathy; age related macular degeneration; chronic inflammatory disorders (e.g., psoriasis and rheumatoid arthritis); and ovarian disorders (e.g., anovulation and infertility, pregnancy loss, ovarian hyperstimulation syndrome, and ovarian neoplasms)). Thus, the present invention provides a method for identifying a disease or disorder associated with aberrant or unwanted 4F2 antigen expression or activity in which a test sample is obtained from a subject and a 4F2 antigen (e.g., mRNA or genomic DNA) is detected, wherein the presence of a 4F2 antigen is diagnostic for a subject having or at risk of developing a disease or disorder associated with aberrant or unwanted 4F2 antigen expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 4F2 antigen expression or activity. For example, such methods can be used to determine whether a subject can be effectively treated with an agent for a disorder associated with unwanted endothelial cell growth. Thus, the present invention provides methods for determining whether a subject can be effectively treated with an agent for a disorder associated with aberrant 4F2 antigen expression in which a test sample is obtained and 4F2 antigen expression is detected (e.g., wherein the abundance of 4F2 antigen expression is diagnostic for a subject that can be administered the agent to treat a disorder associated with aberrant 4F2 antigen expression).

The methods described herein may be performed, for example, by utilizing prepackaged diagnostic kits comprising at least one antibody reagent described herein, which may be conveniently used, e.g., in clinical settings to diagnose patients exhibiting symptoms or family history of a disease or illness involving a 4F2 antigen.

Furthermore, any cell type or tissue in which 4F2 antigen is expressed may be utilized in the prognostic assays described herein.

This invention is further illustrated by the following examples which should not be construed as limiting. The contents of all references, patents and published patent applications cited throughout this application, as well as the Figures are incorporated herein by reference.

### EXAMPLES

### Example 1

### Generation of Antibodies Against Vascular Endothelial Cells

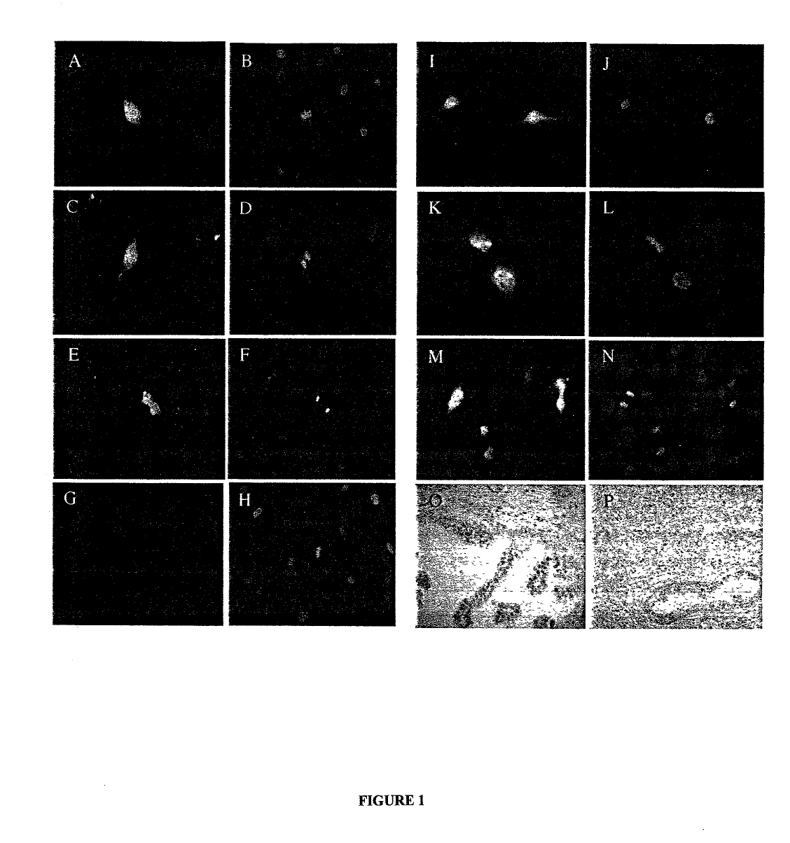

Hybridomas were produced by fusion of myeloma cells with spleen cells from mice immunized with a plasma membrane and cytoplasmic lysate derived from microvascular cell cultures. Antibodies from individual clones were screened by immunofluorescence for reactivity with the cell surface of non-permeabilized endothelial cells and pericytes. Antibodies from several hybridoma clones exhibited cell surface staining. One particular clone, designated βE11, produced an antibody that intensely stained the cell surface of only a subset of endothelial cells (FIGS. 1A-1H). Closer inspection revealed that the βE11 antibody was localized to the surface of mitotic endothelial cells. Anti-βE11 strongly stained the surface of endothelial cells in metaphase, anaphase, and cytokinesis, but not the surrounding interphase cells (FIGS. 1A, 1C, and 1E). In permeabilized endothelial cells, the same distinction could be made. Mitotic cells were intensely stained by anti-βE11, while interphase cells were not (data not shown). Control mouse IgG demonstrated only background staining (FIGS. 1G and 1H).

The βE11 hybridoma cell line was initially grown in DMEM supplemented with 10% fetal bovine serum. The serum was then removed and the cell line was propagated in serum-free medium (Gibco). βE11 antibody was obtained from hybridoma conditioned medium by precipitation with ammonium sulfate. Murine 4F2 antibody was obtained from the conditioned medium of hybridoma clone 4F2C13 purchased from American Type Culture Collection (Manassas, Va.) and maintained according to the manufacturer's instructions. Affinity isolated anti-βE11 and anti-4F2 antibodies were obtained as described below. Purified mouse IgG, (kappa light chain) was obtained from Zymed (So. San Francisco, Calif.) or from Sigma (St. Louis, Mo.). Goat anti-mouse-TRITC was purchased from Zymed or Jackson Immunoresearch (West Grove, Pa.), and goat anti-mouse IgG-HRP was purchased from Chemicon (Temecula, Calif.). Goat anti-mouse IgG(F_{c}) was purchased from Jackson Immunoresearch.

50 mL of five to nine day βE11 conditioned medium was incubated with 1.5 mg goat anti-mouse IgG(F_{c}) conjugated to 7.5 mL Sepharose 4B (Sigma, St. Louis, Mo.) overnight at 4° with constant rotation. The following day, the antibody-Sepharose complexes were poured into a 2.6 cm-diameter column and were washed with 150 mL PBS-azide. Antibody was eluted with 200 mM glycine, pH 2.8, and was collected directly into 1/10 volume 1.0 M Tris, pH 8.0. Typically, 20 mL of glycine was eluted into 2 mL of Tris. The eluate was dialyzed against two exchanges of PBS-azide overnight and was concentrated approximately 100-fold using 10 kDa cutoff centrifuge filters (Millipore, Bedford, Mass.). Antibody concentration was measured by ELISA, and antibodies were stored at 4°.

### Example 2

### Analysis of the βE11 Antigen

A. Analysis in Endothelial Cells

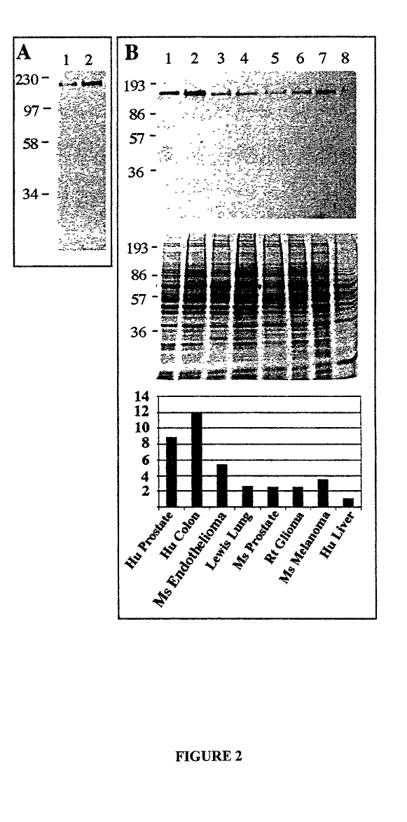

The endothelial cell antigen recognized by anti-βE11 was determined to be approximately 190 kDa by Western blotting (FIG. 2A). Interestingly, subconfluent proliferating vascular endothelial cells expressed a higher level of the βE11 antigen by Western blotting than did confluent, quiescent endothelial cells growth arrested in a monolayer. By densitometric analysis of the Western blot, the level of βE11 antigen in proliferating endothelial cells is approximately three-fold higher than in control growth-arrested cells (data not shown). These results confirmed the immunofluorescence data, indicating that mitotic or proliferating endothelial cells express a higher level of the βE11 antigen than do interphase cells (FIGS. 1A-1F).

B. In Vitro Analysis in Tumor Cells

Because the βE11 antigen was highly expressed on cells undergoing mitosis, it was hypothesized that cells with a high mitotic index, such as tumor cells, would possess abundant cell surface βE11 antigen. The localization of the βE11 antigen in tumor cells (FIGS. 1I-1N) parallels that observed in mitotic vascular endothelial cells. By immunofluorescence, nonpermeabilized LNCaP tumor cells demonstrated a bright, uniform staining, while permeabilized cells revealed a reticular meshwork pattern which covered the entire surface of the cell (FIGS. 1I-1L). Interestingly, no difference was observed in the localization patterns of the βE11 antigen between LNCaP tumor cells in M-phase (FIG. 1K), and those in interphase (FIG. 1I). Other cell lines, such as the 3T3 fibroblast and B16 BL6 melanoma cell lines, demonstrated a similar immunofluorescence pattern. Another tumor cell line, the C2 myoblast cell line, demonstrated mitosis-specific expression of the βE11 antigen. In permeabilized mitotic C2 myoblasts, there was a very intense expression of the βE11 antigen which may be associated with spindle microtubules or centrosomes (FIG. 1M). Interphase cells, in contrast, demonstrated very faint βE11 expression in punctate form around the nucleus. The C2 myoblasts were the only tumor cells studied that demonstrated such mitosis-specific immunolocalization of the βE11 antigen.

C. In Vivo Analysis in Tumor Cells

The βE11 antigen was also localized to regions of prostatic tumor tissue. Anti-βE11 recognized abundant antigen expression in regions of displastic prostate epithelium (FIG. 1O). The surrounding stroma and smooth muscle layers showed only background staining, and control mouse IgG antibody (FIG. 1P) detected no antigen in the tissue sections. Areas of benign prostatic hyperplasia were only weakly stained by anti-βE11. Therefore, these results indicate that the βE11 antigen defines a highly displastic cell population in vivo.

D. Western Blot Analysis in Tumor Cells

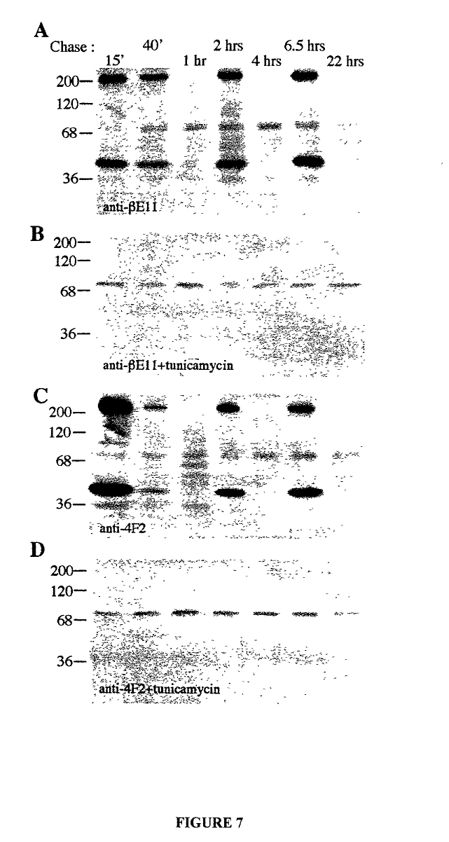

Reducing SDS-PAGE and Western blotting of detergent-solubilized cells indicated that in all tumor cell lines examined, the βE11 antibody recognized an antigen of approximately 190 kDa (FIG. 2B), similar to that recognized in endothelial cells (FIG. 2A). In FIG. 2B, the expression levels of the βE11 antigen in various cell lines were normalized to the total cell protein loaded on the gel. Because anti-βE11 immunoprecipitated an antigen from lysates of tumor cells treated with tunicamycin (an inhibitor of N-linked glycosylation) (set forth in FIG. 7), it is unlikely that the βE11 antibody recognizes a carbohydrate antigen shared by several proteins of varied molecular weights. Therefore, these results indicate that anti-βE11 recognizes a protein epitope rather than a carbohydrate moiety.

### Example 3

### Inhibition of Cell Growth by Anti-βE11

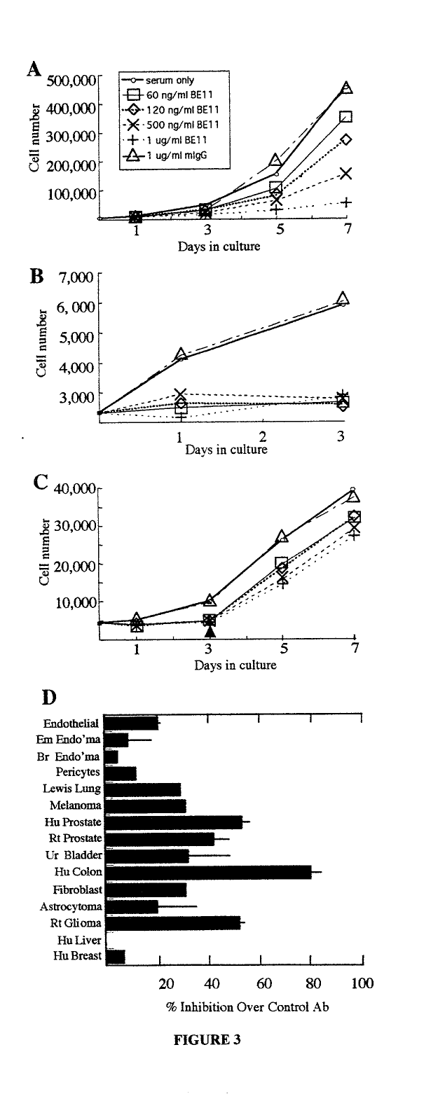

Immunolocalization of βE11 antigen to the surface of nonpermeabilized cells suggested that it would be accessible to its cognate antibody when added to intact, living cells. Because immunofluorescence (FIGS. 1A-1F and 1I-1N) and Western blotting (FIGS. 2A and 2B) indicated that the presence of the βE11 antigen correlated with proliferation, the ability of anti-βE11 to inhibit cell growth in vitro was investigated. Primary cells, such as vascular endothelial cells and pericytes, as well as established tumor lines, such as Lewis lung, LNCaP human prostate, and B16 BL6 murine melanoma lines, were grown in vitro in the presence or absence of the βE11 antibody or an isotype matched control. Anti-βE11 inhibited the proliferation of many different tumor cells, such as those derived from human colon (80% inhibition; FIG. 3A) and human prostate (55% inhibition; FIG. 3B). However, anti-βE11 demonstrated little inhibition (15-20%) in primary cells, and no inhibition in certain tumor cell lines such as HEPG2 hepatocarcinoma (FIG. 3D). Anti-βE11 thus has varied effects on the growth of different cell types in vitro. The inhibitory effect of the βE11 antibody is dose dependent with maximal inhibition at 0.5-1.0 μg/mL for all cell lines tested. Anti-βE11 was determined not to be cytotoxic as gross inspection of the inhibited cells by phase contrast microscopy showed no evidence of necrosis or apoptosis (data not shown). Furthermore, when the antibody was removed, cell proliferation resumed, indicating that the inhibitory effect of anti-βE11 was reversible (FIG. 3C).

Interestingly, in some cell lines, the expression level of the βE11 antigen by Western blotting corresponded to inhibitory potential of the βE11 antibody in the in vitro growth assays. For example, the human colon carcinoma and the LNCaP human prostate tumor cells expressed abundant antigen (FIG. 2B) and were also significantly inhibited in growth by the βE11 antibody (FIGS. 2B and 2C). However, rat glioma cells, which were inhibited to the same extent as the LNCaP tumor cells (approximately 55% inhibition), were determined to express approximately 28% as much antigen as the LNCaP cells.

### Example 4

### Identification of the Cell-Derived βE11 Antigen

A. Two-Dimensional SDS-PAGE and Spectrometry

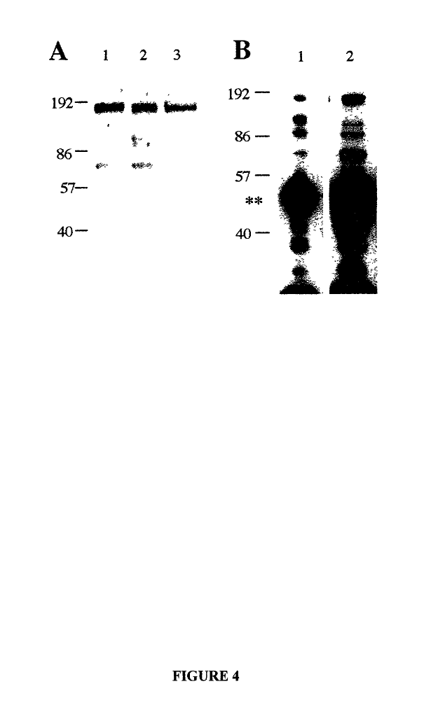

In order to circumvent problems of low abundance or N-terminal blockage commonly observed in protein sequence determinations of cell-surface antigens, the βE11 antigen was identified using two-dimensional gel electrophoresis and mass spectrometry. This technique is approximately 100-1,000 times as sensitive as Edman degradation and can yield sequence data from fmol amounts of digested peptide. Therefore, even if the βE11 antigen was both N-terminally blocked and present in very low quantities in cells, mass spectrometry should yield sequence data for it. To avoid possible contaminating proteins from serum, a cell line grown in the absence of serum was utilized as a source for the βE11 antigen. Because the B16 BL6 melanoma was the only cell line that could be propagated in the absence of serum and because it expressed significant βE11 antigen as determined by Western blotting (FIG. 2B), it was used as a cellular source of the βE11 antigen. Anti-βE11 recognized its antigen in detergent lysates of melanoma cells grown in the absence of serum virtually to the same extent as it recognizes its antigen in cell lysates derived from cells grown in the presence of serum (FIG. 4A). Furthermore, anti-βE11 immunoprecipitated an antigen of approximately 190 kDa (as well as smaller molecular weight species) from serum-free melanoma cell lysates (FIG. 4B).

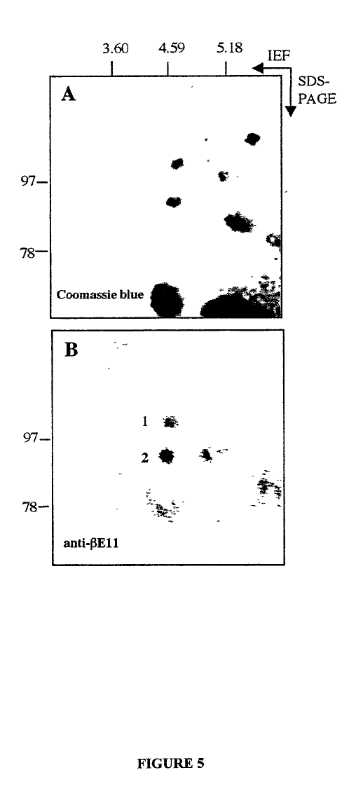

Proteins derived from a detergent-solubilized extract of melanoma cells grown in the absence of serum were separated by isoelectric focusing from pH 3 to 7 in the first dimension and then by SDS-PAGE in the second dimension. As shown in FIG. 5, two individual spots, both having a pI of approximately 4.6 and molecular weights of approximately 110 kDa and 93 kDa, were determined by Western blotting to be immunoreactive with anti-βE11. Both spots were identified by Coomassie blue staining of the corresponding gel and were excised and analyzed by mass spectrometry. The 110 kDa spot generated sequence for calnexin, an endoplasmic reticulum-resident membrane protein which transiently associates with many proteins shortly after synthesis, and CBP-140, a heat shock protein homolog. The 93 kDa spot generated sequence for calnexin and the 4F2 cell surface antigen heavy chain. Interestingly, the 4F2 antigen is a 125 kDa cell surface glycoprotein complex which was originally characterized to be a protein highly expressed on activated vs. resting lymphocytes. Subsequently, this complex was determined to be a protein complex that is expressed in all proliferating cells.

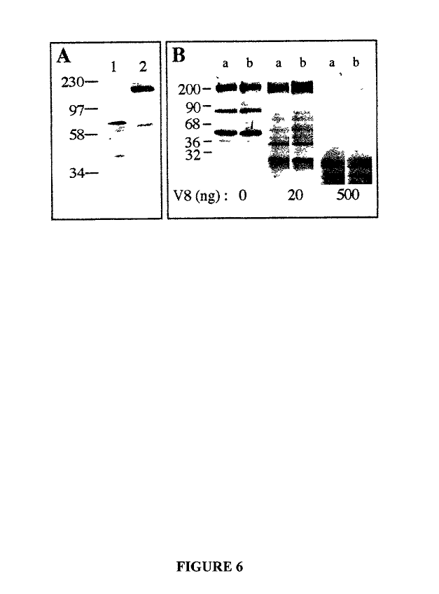

Because of the similarities of the 4F2 complex to the βE11 antigen in molecular weight, cell surface localization, and possible function in cell growth, it is likely that the two antigens are identical or closely related family members. To determine whether anti-βE11 and anti-4F2 recognize the same cell-derived antigen, staining patterns of anti-4F2 and anti-βE11 on Western blots were directly compared. In lysates of serum-free B16 BL6 melanoma cells, anti-βE11 recognizes an antigen most strongly at approximately 190 kDa and to a lesser extent an antigen at approximately 75 kDa (FIG. 6A). Under the same conditions, anti-4F2 primarily recognized an antigen at approximately 75 kDa. Therefore, although antigens of similar molecular weights were identified by both antibodies on Western blots, the two antibodies also recognized distinct molecular weight species.

The differences observed in Western blots probed with anti-βE11 vs. anti-4F2 raised the possibility that anti-βE11 recognized a protein that is similar to 4F2 but may be a distinct family member. In order to determine whether the βE11 antigen is distinct from 4F2, limited protease digests were performed on antigens immunoprecipitated by both antibodies. B16 BL6 melanoma serum-free cells were metabolically labeled. The proteins immunoprecipitated by anti-βE11 and anti-4F2 were subjected to limited V8 protease digestion. Both anti-βE11 and anti-4F2 immunoprecipitate antigens of the same molecular weight (FIG. 6B). Furthermore, the fingerprints of peptides generated by V8 protease digestion of immunoprecipitated proteins were identical for both βE11 and 4F2 antigens. Lane 7 shows that the proteins digested with V8 protease are specifically recognized by antibodies to βE11 and 4F2 because they are not immunoprecipitated by a mouse IgG control. These results suggest that at this resolution, the βE11 antigen is identical to the 4F2 heavy chain.

B. βE11 and 4F2 Antigens in the Absence of N-Linked Glycosylation

In order to determine whether both anti-βE11 and anti-4F2 recognize similar antigens in the absence of N-linked glycosylation, pulse-chase analysis of serum-free B16 BL6 melanoma cells in the presence of tunicamycin, an inhibitor of N-linked glycosylation, was performed. Melanoma cells grown in serum-free medium were pulsed for 2 hours with ³⁵S methionine, and after chasing with ³⁵S-free complete medium for various times, the cells were lysed. Labeled proteins immunoprecipitated by anti-βE11 and anti-4F2 were directly compared in FIG. 7. At all timepoints of chase tested, a single band of approximately 80 kDa was immunoprecipitated by both antibodies. Interestingly, the 200 and 45 kDa bands immunoprecipitated by both anti-βE11 and anti-4F2 in the absence of tunicamycin (FIG. 6B, lanes 1 and 2) were not immunoprecipitated in the presence of this inhibitor (FIG. 7). The similarity between the metabolically-labeled antigens immunoprecipitated by both antibodies confirmed the protease fingerprint analysis (FIG. 6B) which indicates that the βE11 and 4F2 antigens are identical.

C. Discussion

1. Functional Definition of the βE11 Antigen

Antigens that are highly expressed on tumor cells but are absent or expressed very little on normal cells have previously been characterized, but no correlation between these antigens and processes inherent to tumor cell growth has been made. These examples indicate that expression of the βE11 antigen is functionally linked to a fundamental aspect of tumor cells because it is found on most cells that are undergoing mitosis or are growing, and a hallmark of tumor cells is their capacity for uncontrolled growth.

Endothelial cells undergoing mitosis showed robust staining with anti-βE11 (FIGS. 1A-1F), and high expression of the βE11/4F2 antigen defined a cell population that was actively proliferating vs. one that was growth arrested and quiescent (FIG. 2A). Several authors have reported cell cycle-specific expression of 4F2 protein and mRNA, but controversy exists as to the exact timing. Various studies report 4F2 protein expression peaking in early G1 and 4F2 mRNA levels peaking during S phase or remaining constant throughout the cell cycle after rising sharply 3-6 hours following serum stimulation. These examples indicate that high M-phase expression of the βE11 antigen could be due to transport of intracellular βE11/4F2 pools to the cell surface or to exposure of a masked or cryptic epitope. However, because anti-βE11 recognizes a protein, not a carbohydrate, moiety (FIG. 7), the M-phase-specific expression of the βE11/4F2 antigen is not due to exposure of an epitope created by glycosylation.

In addition to mitotic endothelial cells, all tumor cells studied exhibit abundant cell-surface expression of the βE11/4F2 antigen (FIGS. 2I-2N). However, unlike endothelial cells, βE11/4F2 antigen expression appears similar in mitotic and interphase tumor cells. The 4F2 antigen was originally discovered on activated, but not resting B and T cells, but was later observed on all human tissue culture cell lines tested. Interestingly, it has been suggested that a difference in the organization of the 4F2 antigen in the plasma membrane is responsible for high expression of the 4F2 antigen in neoplastic and embryonic vs. normal adult cells, and it has been proposed that overexpression of the 4F2 antigen results in malignant transformation. The observation presented herein that both mitotic and interphase tumor cells show intense cell surface staining for the βE11/4F2 antigen supports these ideas and indicates that tumor cells constitutively express the form of the βE11/4F2 antigen observed in mitotic primary cells. Therefore, anti-βE11 may recognize an intrinsic characteristic of tumor cells, whether in M phase or interphase, that is shared with mitotic primary cells. Moreover, as shown in FIG. 1O, anti-βE11 targets highly displastic regions of prostatic tumor tissue in histological sections and does not stain the surrounding stroma. This is the first demonstration that an antibody which recognizes the 4F2 antigen can identify displastic regions of a solid tumor in tissue sections and suggests that this antibody may be able to target tumors in vivo.

2. Biochemical Characterization of the βE11 Antigen

SDS-PAGE and Western blot analysis indicated that the major antigen recognized by anti-βE11 in tumor cell lysates is approximately 190 kDa (FIG. 2B). However, three major antigens were immunoprecipitated by both anti-βE11 and anti-4F2 from metabolically-labeled B16 BL6 melanoma cells (FIG. 7). One is an approximately 80 kDa doublet, another is a 200 kDa species, and the third is a 45 kDa antigen. The 200 and 45 kDa species appear cyclically during the 22-hour chase period while the 69 kDa/80 kDa doublet is fairly constant throughout.

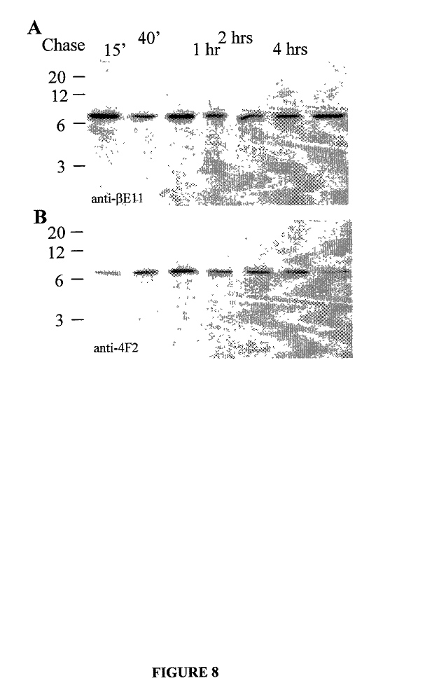

The primary structure of the murine 4F2 heavy chain predicts a 526 amino acid type II glycoprotein (Parmacek et al. (1989) Nucl. Acid Res. 17:1915-1931), and the 4F2 antigen has been well characterized to be a disulfide-linked complex of 125 kDa which resolves into an 85 kDa heavy chain and a 40 kDa light chain on reducing SDS-PAGE in lymphoid cells (Chowdhury et al. (1998) Proc. Natl. Acad. Sci. USA 95:669-674; Hunkapiller et al. (1984) Nature 310:105-111). In fibroblasts, the 4F2 heavy chain is a doublet of 73/85 kDa in fibroblasts (Azzarone et al. (1985) Exp. Cell Res. 159:451-462). The approximately 80 kDa doublet may represent non-glycosylated βE11/4F2 heavy chain that remains unassociated with the 45 kDa light chain. Perhaps this pool of heavy chain is sequestered by calnexin because calnexin peptides were discovered in the mass spectrometry analysis of two-dimensional gel spots immunoreactive with anti-βE11 (FIG. 5). The other pool of βE11/4F2 antigen is the 200 kDa antigen which may represent a non-reduced oxidative complex of glycosylated heavy chain and light chain that incorporates both species of the heavy chain doublet. N-linked glycosylation is necessary for the appearance of the 200 kDa antigen and its association with the 45 kDa light chain because in tunicamycin-treated cells only a single antigen of approximately 80 kDa is immunoprecipitated by anti-βE11 and anti-4F2 throughout the chase period (FIG. 8).

It is possible that creation of the 200 kDa and 45 kDa antigen complex exposes an epitope that is identified by anti-βE11 and anti-4F2 more efficiently than the epitope on the approximately 80 kDa heavy chain doublet. The 200 kDa and 45 kDa antigens are more abundant than both antigens in the ˜80 kDa doublet (FIG. 6). In primary endothelial cells, exposure of this epitope may account for the higher immunoreactivity with anti-βE11 in mitotic vs. primary cells.

The 200 kDa βE11/4F2 antigen may be shed of the from the cell surface. A 200 kDa radiolabeled antigen accumulates over time in both anti-βE11 and anti-4F2 immunoprecipitates of 0.2-μm filtered cultured supernatants from metabolically-labeled B16 BL6 melanoma cells (data not shown). Shedding of the βE11 antigen may be important in vivo as soluble βE11 antigen could be a diagnostic tool for monitoring tumor progression in cancer.

3. Inhibition of Cell Growth

The 4F2 antigen targeted by anti-βE11 is likely to play an important role in regulating cell proliferation because anti-βE11 inhibits cell growth in vitro (FIG. 3). Gross examination of the inhibited cells by phase contrast microscopy revealed that the antibody did not induce apoptosis or necrosis (R. Nayak, unpublished personal communication). Instead, it reversibly impeded proliferation, and the growth-inhibited cells did not appear to be arrested at a particular phase of the cell cycle.

The 4F2 antigen can transport neutral and cationic amino acids. Because anti-βE11 inhibits tumor cell growth, transport of certain amino acids by the βE11/4F2 antigen may be necessary for cell proliferation. Early reports have demonstrated that significant amino acid transport occurs in mitosis as well as in interphase. Anti-βE11 probably inhibits amino acid transport during interphase and possibly mitosis. The gross observation that cells inhibited in growth by anti-βE11 do not arrest at a certain phase of the cell cycle suggests that amino acid transport mediated by the βE11/4F2 antigen is not limiting for cell cycle progression and growth. Therefore, anti-βE11 may reversibly inhibit cell growth because it impedes transport of necessary nutrients into the cell.

Previous work has demonstrated that antibodies to 4F2-like molecules reversibly inhibit bladder cancer and T-lymphoma cell growth in vitro in a dose-dependent manner. Antibody concentrations used in these studies ranged from 1-5 μg/ml, at which inhibition was minimal, to a maximally inhibitive dose of 100 μg/ml. Other antibodies to 4F2 impede the growth of fibrosarcoma cells and block DNA synthesis in activated peripheral blood mononuclear cells. These examples indicate that an antibody which recognizes 4F2 not only inhibits tumor cell growth in vitro but is also capable of recognizing mitotic vs. interphase cells. These data, therefore, provide novel insight into how the 4F2 antigen is associated with proliferating and activated cells. High cell-surface expression during mitosis links it to a process inherent to dividing cells.

Antibody targeting of cell-surface molecules other than 4F2, such as disialoganglioside G_{D3}, the receptors for IL-2, transferrin and EGF, and HLA-DR, also inhibits cell growth in vitro and in vivo. Significant drawbacks to these studies, however, are the high concentrations of antibody needed to inhibit cell proliferation (6-50 μg/mL for anti-HLA-DR), the limited cell-type specificity of the antibodies, and harmful in vivo side effects. Anti-βE11, on the other hand, significantly inhibits the growth of a variety of tumor cells at sub-microgram/ml doses. Furthermore, anti-βE11 inhibits the growth of primary endothelial cells in culture, albeit to a limited extent. A hallmark of many growing tumors is extensive angiogenesis, a process characterized by proliferation of endothelial cells that extend the vascular network so that nutrients may be effectively delivered to rapidly dividing tumor cells. Anti-βE11 alone or conjugated to a toxin may not only inhibit the growth of tumor cells but may also attenuate angiogenesis. Therefore, in terms of tumor therapeutics, anti-βE11 may have distinct advantages over other monoclonal antibodies that have been used to inhibit tumor cell growth.

4. Identity With 4F2

4F2 antigens belong to a family of heterodimeric transmembrane proteins expressed on the surface of activated cells that are composed of different light chain subunits, of which 6 have been identified, disulfide-bonded to a common heavy chain. FIG. 6B indicates that limited protease digest fingerprints of anti-βE11 and anti-4F2 immunoprecipitates are the same. Therefore, at this level of resolution, both heavy and light chains of the βE11 and 4F2 antigens are identical in these cells.

The differences between staining patterns of Western blots probed with anti-βE11 and anti-4F2 (FIG. 6A) may be attributed to differences in affinity for various forms of the 4F2 antigen. Anti-βE11 may have a higher affinity for the complex of 4F2 heavy and light chain at approximately 200 kDa, and anti-4F2 could have a higher affinity for the uncomplexed 4F2 heavy chain at 75 kDa. On a Western blot, where the incubation time of antibody with antigen is only 3-6 hours, these differences in affinity are apparent. However, in the immunoprecipitations, where antibody is incubated with antigen overnight (14-18 hours), these differences are masked (FIG. 6B) because even low-affinity interactions have time to form.

5. Therapeutic Potential

The βE11 antigen represents an ideal target for tumor immunotherapy. The efficacy of anti-βE11 in tumor therapy is not just limited to directly inhibiting the growth of tumor cells themselves, however. The βE11 antigen was first discovered on mitotic endothelial cells. Another hallmark of many growing tumors is extensive angiogenesis. This process is characterized by proliferation of endothelial cells that extend the vascular network so that nutrients may be effectively delivered to rapidly dividing tumor cells. Anti-βE11 inhibits the growth of primary endothelial cells in culture (FIG. 3), albeit to a limited extent. Thus anti-βE11 alone or conjugated to a toxin may not only inhibit the growth of tumor cells but may also attenuate angiogenesis. With such a dual function, anti-βE11 can effectively halt the spread of a tumor.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of identifying a precancerous cell, comprising contacting a precancerous cell with an antibody that binds to a 4F2 antigen present on a precancerous cell, wherein binding of the antibody to a cell identifies the cell as a precancerous cell.

2. The method of claim 1, wherein the precancerous cell is obtained from a biological sample.

3. The method of claim 2, wherein the biological sample is selected from the group consisting of tissue, cells, blood, serum, urine, feces, and saliva.

4. The method of claim 1, wherein the precancerous cell is obtained from a biopsy sample.

5. The method of claim 1, wherein the antibody is a monoclonal antibody.

6. The method of claim 5, wherein the monoclonal antibody is anti-βE11.

7. A method of distinguishing mitotic cells from cells in interphase comprising,
i) contacting a population of cells comprising at least one mitotic cell and at least one cell in interphase with an antibody that binds to a 4F2 antigen, wherein binding of the antibody to a cell identifies the cell as a mitotic cell;
ii) detecting the binding of the antibody to a cell of the cell population; and
iii) identifying the cell that binds the antibody as a mitotic cell to thereby distinguish mitotic cells from cells in interphase.

8. The method of claim 7, wherein the cells are obtained from a biological sample.

9. The method of claim 8, wherein the biological sample is selected from the group consisting of tissue, cells, blood, serum, urine, feces, and saliva.

10. The method of claim 7, wherein the cells are obtained from a biopsy sample.

11. The method of claim 7, wherein the antibody is a monoclonal antibody.

12. The method of claim 11, wherein the monoclonal antibody is anti-βE11.

13. A method of detecting displastic regions of a tumor comprising contacting a tumor with an antibody that binds to a 4F2 antigen such that displastic regions of a tumor are detected.

14. The method of claim 13, wherein the tumor is obtained from a biological sample.

15. The method of claim 13, wherein the tumor is obtained from a biopsy sample.

16. The method of claim 13, wherein the antibody is a monoclonal antibody.

17. The method of claim 16, wherein the monoclonal antibody is anti-βE11.

18. A method of inhibiting proliferation of a precancerous cell comprising contacting a precancerous cell with an antibody that binds to a 4F2 antigen such that proliferation of a precancerous cell is inhibited.

19. The method of claim 18, wherein the antibody is a monoclonal antibody.

20. The method of claim 19, wherein the monoclonal antibody is anti-βE11.

21. The method of claim 18, wherein the antibody is administered to a subject in a pharmaceutically acceptable formulation.

22. The method of claim 21, wherein the subject is human.

23. A method of inhibiting proliferation of a precancerous cell comprising contacting a precancerous cell with an antibody that binds to a 4F2 antigen and a low molecular weight inhibitor such that proliferation of a precancerous cell is inhibited.

24. The method of claim 23, wherein the antibody is a monoclonal antibody.

25. The method of claim 24, wherein the monoclonal antibody is anti-βE11.

26. The method of claim 23, wherein the antibody is administered to a subject in a pharmaceutically acceptable formulation.

27. The method of claim 26, wherein the subject is human.

28. A method of treating a disorder associated with endothelial cell growth comprising contacting an endothelial cell with an antibody that binds to a 4F2 antigen such that the disorder is treated.

29. The method of claim 28, wherein the antibody is a monoclonal antibody.

30. The method of claim 29, wherein the monoclonal antibody is anti-βE11.

31. The method of claim 28, wherein the antibody is administered to a subject in a pharmaceutically acceptable formulation.

32. The method of claim 31, wherein the subject is human.

33. The method of claim 28, wherein the disorder is selected from the group consisting of diabetic retinopathy, age related macular degeneration, chronic inflammatory disorders, and ovarian disorders.

34. A method of inhibiting tumor derived angiogenesis comprising contacting an endothelial cell with an antibody that binds to a 4F2 antigen such that proliferation of an endothelial cell is inhibited.

35. The method of claim 34, wherein the antibody is a monoclonal antibody.

36. The method of claim 35, wherein the monoclonal antibody is anti-βE11.

37. The method of claim 34, wherein the antibody is administered to a subject in a pharmaceutically acceptable formulation.

38. The method of claim 37, wherein the subject is human.

39. A method of detecting the presence of tumor cells in a patient comprising testing for the presence of a 4F2 antigen in a blood sample of the patient.

40. The method of claim 39, wherein the tumor cells are prostate tumor cells.

41. The method of claim 39, wherein the tumor cells are selected from the group consisting of colon tumor cells, gliomas, melanomas, fibroblast tumor cells, breast tumor cells, and liver tumor cells.

42. A kit for diagnosing and/or prognosing prostate cancer comprising an antibody that recognizes the 4F2 antigen and instructions for use.

43. A method for facilitating the prognosis of a disorder associated with endothelial cell growth in a subject, comprising:
i) obtaining a biological sample from a subject; and
ii) detecting a 4F2 antigen with an antibody that binds to a 4F2 antigen, thereby facilitating the prognosis of a disorder associated with endothelial cell growth in a subject.

44. The method of claim 43, wherein the biological sample is selected from the group consisting of tissue, cells, blood, serum, urine, feces, and saliva.

45. The method of claim 43, wherein the sample is obtained from a biopsy sample.

46. The method of claim 43, wherein the antibody is a monoclonal antibody.

47. The method of claim 46, wherein the monoclonal antibody is anti-βE11

48. The method of claim 43, wherein the disorder is selected from the group consisting of diabetic retinopathy, age related macular degeneration, chronic inflammatory disorders, and ovarian disorders.

49. The antibody anti-βE11.
